(19) United States
(12) Patent Application Publication
(10) Pub. No.: US 2012/0123498 A1
(43) Pub. Date: May 17, 2012

(54) SLEEP APNEA TREATMENT SYSTEM

(75) Inventor: Yossi GROSS, Moshav Mazor (IL)

(73) Assignee: RAINBOW MEDICAL LTD., Herzliya (IL)

(21) Appl. No.: 12/946,246

(22) Filed: Nov. 15, 2010

(51) Int. Cl. A61N 1/36

(52) U.S. Cl. 607/42

(57) ABSTRACT

An apparatus is provided for treating obstructive sleep apnea. The apparatus has an external device containing a breathing sensor to sense activity associated with breathing of a subject, and also containing a control unit, coupled to the breathing sensor, for generating a signal in response to anatomical activity associated with breathing. The apparatus also has a sublingual implant, containing a receiver, electrodes and circuitry, which wirelessly receives a signal from the external device and applies current to a sublingual muscle site in response to the signal, in a plurality of successive periods of inspiration, even in the absence of a detection of apnea by the external device.

### FIELD OF EMBODIMENTS OF THE INVENTION

Some applications of the present invention generally relate to implanted medical apparatus. Specifically, some applications of the present invention relate to apparatus and methods for treating sleep apnea, particularly, obstructive sleep apnea (OSA).

### BACKGROUND

Sleep apnea is a chronic sleep breathing disorder typically characterized by abnormal pauses (apneas) in an individual's breathing, or alternatively, by instances of abnormally low breathing. Each apnea event can range in duration from seconds to minutes. Sleep apnea, although often unrecognized for years by the subject herself, can have long term health effects: Sleep apnea and other sleep breathing disorders are associated with cardiovascular disease, myocardial infarction, high blood pressure, stroke, arrhythmias, diabetes, sleep-deprived driving accidents, and cerebrovascular disease.

In general, there are three forms of sleep apnea: central sleep apnea (CSA), obstructive sleep apnea (OSA), and complex sleep apnea—a heterogeneous group of sleep-related breathing disturbances, with characteristics partially related to both CSA and OSA.

With CSA, cessations in air flow occur without respiratory effort. With OSA (alternatively, obstructive sleep apnea/hypopnea syndrome (OSAHS)), which has overlapping pathogenesis and clinical presentation with CSA, a subject's breathing is interrupted by a physical blockage in the airflow, often characterized, but not limited to snoring and restless sleep. Cardiac output also tends to decrease during apnea events. Snoring, along with other risk factors such as obesity, diabetes or smoking, is often indicative of OSA, particularly when snoring is punctuated by deep gasps.

OSA, the most common form of sleep apnea, is characterized by repetitive collapse and reopening of the upper airway during sleep, resulting in complete or partial blockage of the upper airway during sleep and leading to hypoxemia and hypercapnia. Typically, dilator muscles in the subject's airway stiffen and dilate various regions of the upper airway while the subject is awake. This activity, however, is reduced during sleep, effectively narrowing the upper airway. Of the dilator muscles, the largest is the genioglossus, (the muscle that forms the majority of the tongue).

Current treatments includes positive airway pressure (PAP) therapy, particularly, continuous positive expiratory pressure (CPAP), oral appliances, surgery, including genioglossus advancement, tongue radiofrequency, midline glossectomy, hyoid suspension, maxillomandibular advancement, and lifestyle changes such as positional therapy and weight loss.

### SUMMARY OF EMBODIMENTS

For some applications of the invention, apparatus for treating obstructive sleep apnea comprises a breathing sensor for sensing breathing activity, configured for placement outside of a body of a subject. Breathing can be sensed by, for example, sensing electrical activity, breathing-related motions, or via video imaging of the subject's respiration. The apparatus typically also has a control unit, coupled to the breathing sensor, configured to generate signals in response to the sensed activity associated with breathing. The apparatus also includes an implant, typically a sublingual implant, typically comprising a wireless receiver for receiving the signals, two or more electrodes, circuitry coupled to the electrodes, and a coil coupled to the circuitry to wirelessly receive power from the external device. The circuitry drives the electrodes to apply current to a sublingual muscle site of the subject in response to the signal.

There is therefore provided, in accordance with an application of the present invention, apparatus for treating obstructive sleep apnea, including:

an external device, including:
a breathing sensor configured for placement outside of a body of a subject and to sense activity associated with breathing of the subject;
a control unit, coupled to the breathing sensor, configured to generate a signal in response to the sensed activity associated with breathing; and

a sublingual implant, including a wireless receiver for receiving the signal, two or more electrodes, and circuitry coupled to the electrodes, the circuitry configured to drive the electrodes to apply current to a sublingual muscle site of the subject in response to the signal, in a plurality of successive periods of inspiration of the subject, even in the absence of a detection of apnea by the control unit.

For some applications, the breathing sensor is configured to sense a parameter selected from the group consisting of: airway pressure, snoring sounds, snoring motions, mechanical motion, and electrical activity associated with respiration.

For some applications, the sublingual implant is operative to vary at least one parameter of the applied current, the parameter selected from the group consisting of: a frequency of pulses of the current, an intensity of pulses of the current, and a duration of pulses of the current.

For some applications, the sublingual implant includes an implant sensor configured to provide feedback to the external device.

For some applications, the control unit is configured to generate the signal during respective inspiratory phases of at least 30% of respiratory periods of the subject in a ten-minute period.

There is further provided, in accordance with an application of the present invention, a method for treating obstructive sleep apnea, including:

identifying a subject as suffering from obstructive sleep apnea; and

in response, implanting, in a sublingual muscle of the subject, a sublingual implant that is configured to:
directly stimulate the muscle in a plurality of successive periods of inspiration of the subject, even in the absence of a detection of apnea; and
wirelessly receive energy from an external device to power the sublingual implant.

For some applications, the method includes configuring the external device to sense activity associated with breathing of the subject, and to generate a signal that causes the implant to stimulate the muscle during the inspiration periods.

For some applications, configuring the external device further includes placing the external device under a mattress.

For some applications, the method includes configuring the external device to sense breathing of the subject and to provide a signal to the sublingual implant to cause the implant to directly stimulate the muscle in response to the sensing of the breathing by the external device, during respective inspiratory phases of at least 30% of respiratory periods in an hour.

For some applications, implanting the sublingual implant includes sublingually injecting the implant.

There is still further provided, in accordance with an embodiment of the present invention, a method for treating obstructive sleep apnea, including:

sublingually implanting a sublingual implant into a subject; and

configuring an external device to:
sense breathing of the subject;
provide a signal to the implant to cause the implant to directly stimulate a muscle in response to the sensing of the breathing by the external device, even in the absence of a detection of apnea, during respective inspiratory phases of at least 30% of respiratory periods in a ten-minute period; and
wirelessly transmit energy from the external device to the sublingual implant to power the sublingual implant.

There is yet further provided, in accordance with an embodiment of the present invention, a method for treating obstructive sleep apnea, including:

sublingually implanting a sublingual implant into a subject;

configuring the sublingual implant to provide feedback regarding breathing of the subject to an external device; and
configuring the external device to provide a signal to the implant to cause the implant to stimulate a muscle in response to the feedback provided by the sublingual implant.

For some applications, configuring the external device includes configuring the external device to sense breathing, and to provide the signal to the implant to cause the implant to stimulate the muscle in response to the feedback provided by the sublingual implant and in response to the breathing sensed by the external device.

For some applications, configuring the sublingual implant to provide feedback regarding the breathing of the subject includes configuring the sublingual implant to provide feedback regarding snoring of the subject.

The present invention will be more fully understood from the following detailed description of embodiments thereof, taken together with the drawings, in which:

### BRIEF DESCRIPTION OF THE DRAWINGS

FIG. 1 is a schematic illustration of an external device, in accordance with some applications of the present invention; and

FIG. 2 is a schematic illustration of an implanted device, in accordance with some applications of the present invention.

### DETAILED DESCRIPTION OF EMBODIMENTS

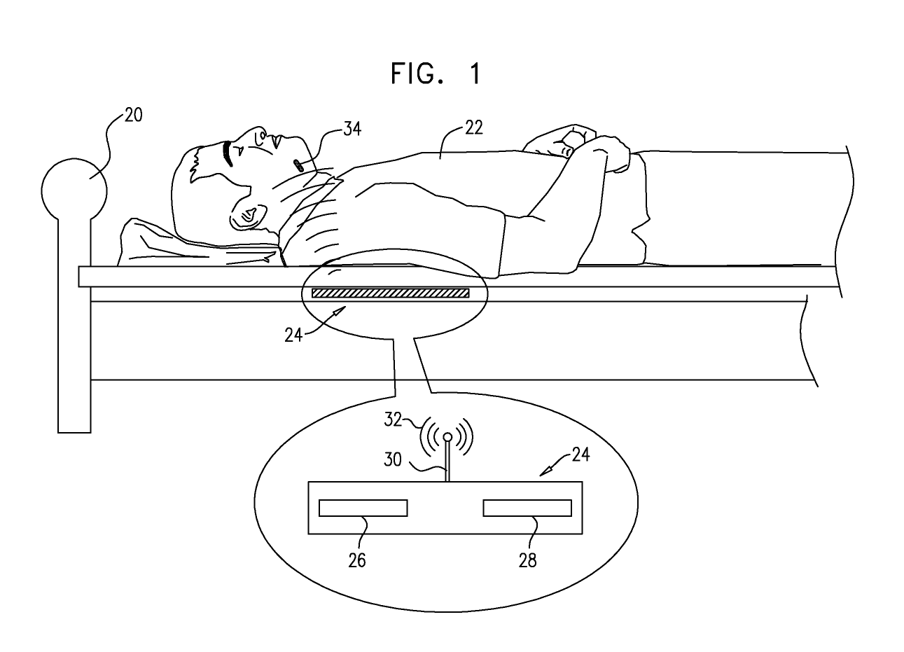

Reference is now made to FIG. 1, which is a schematic illustration of a bed 20 and a subject 22 lying supine in the bed. Typically, the subject is sleeping. A typical external device 24 is shown in detail comprising a control unit 26 and a breathing sensor 28. The breathing sensor senses respiration, and control unit sends signals 32 responsive thereto to a sublingual implant 34 typically implanted in a sublingual muscle of the subject (e.g., the genioglossus muscle). The signals are sent to the implant at a particular temporal point in the subject's breathing pattern, as determined by the breathing sensor. The external device, in accordance with some applications of the present invention, is placed in proximity to the subject under the chest of the subject, or under the subject's pillow or mattress. Alternatively, the external device can be placed anywhere near the subject, such that the sublingual implant receives signals from the external device. One or more antennas 30 of external device 24 are typically configured to send the signals to the implant, as described hereinbelow.

External device 24 typically sends signals 32 to sublingual implant 34, which drive the sublingual implant to stimulate a sublingual muscle (e.g., the genioglossus muscle, not shown). This stimulation is typically in a plurality of successive periods of respiratory inspiration of subject 22, and typically even in the absence of a detection of apnea by the external device. For some applications, signals are provided to the implant to cause the implant to stimulate the muscle at a non-inspiration point, or at multiple points during a respiratory cycle.

In some applications, external device 24 sends signals 32 to sublingual implant 34 to stimulate the muscle for a pre-determined length of time or in a particular pattern, or both, over the course of subject's 22 sleep cycle.

For some applications, periods of no stimulation by sublingual implant 34 are provided, during the course of the sleep cycle, to prevent over-stimulating a muscle (e.g., the genioglossus muscle, not shown).

Typically, signals 32 are provided to stimulate sublingual implant 34 during at least 30% or at least 50% of the respiratory periods over the course of a ten-minute period, even in the absence of a detection of apnea by external device 24.

In addition, the external device 24 is typically configured to only provide signals 32 to the sublingual implant 34 to stimulate a muscle when the subject 22 is sleeping.

External device 24 typically wirelessly powers sublingual implant 34, via signals 32, e.g., radio waves or inductive coupling, as described hereinbelow.

Breathing sensor 28 typically comprises a piezoelectric crystal, a strain gauge a pressure sensor, an accelerometer, an optical sensor, an audio sensor, a video imager, and/or a cardiac sensor, or combinations thereof, to detect, for example, breathing-related motions, snoring, electrical activity, and/or other indications of the subject's breathing. The one or plurality of sensors provide information regarding, but not limited to, the subject's airway pressure, snoring sounds and motions, mechanical motion associated with respiration, and/or electrical activity associated with respiration. In addition, other sensors known in the art (e.g., a blood oxygen saturation sensor) assess the operation of external device 24 and implant 34, and to support closed-loop control of external device 24 and implant 34.

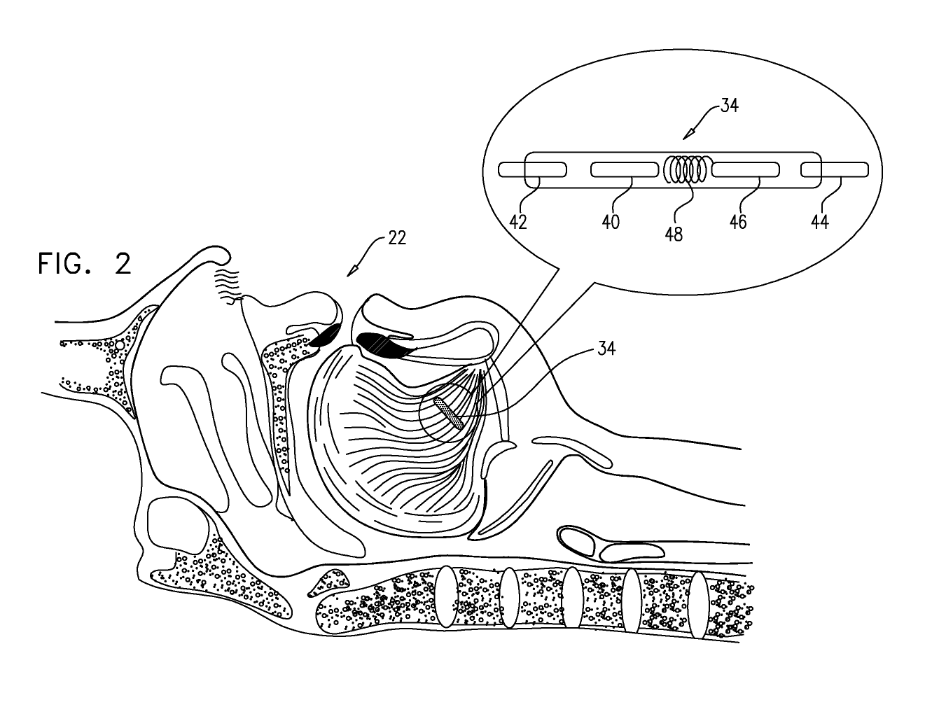

Reference is now made to FIG. 2, which is a schematic illustration of sublingual implant 34 in subject 22, in accordance with some applications of the present invention. Sublingual implant 34 shown in FIG. 2 is typically the sublingual implant described hereinabove with reference to FIG. 1. Implant 34, as shown enlarged, contains a wireless receiver 40, two or more electrodes 42 and 44, and circuitry 46, coupled to the electrodes. The implant and its components are encapsulated using techniques known in the art. Material for the electrodes can be chosen based on numerous criteria, as are known in the art, including, tissue response, allergic response, electrode-tissue impedance, and radiographic visibility.

Circuitry 46 is typically configured to drive electrodes 42 and 44 to apply current, typically as a train of pulses, to a sublingual muscle site of subject 22, in response to signals 32 from external device 24, in a plurality of successive periods of inspiration of the subject. Typically, signals 32 are generated substantially for the duration of any given sleep cycle, even in the absence of a detection of apnea by the external device. Alternatively, signals 32 are generated for only a portion of the sleep cycle, e.g., for at least 30% or at least 50% of the cycle.

It is to be noted that the sublingual implant's particular location in the muscle is shown by way of illustration and not limitation.

In some applications, sublingual implant 34 can include an implant sensor (not shown), such as a vibration sensor sensitive to snoring vibrations, or a motion sensor. The implant sensor allows the implant to operate independently of, or in conjunction with, external device 24, optionally providing feedback to the external device. In other applications, one or more other implant sensors are included in the implant to be used independently by the implant, or in conjunction with the external device.

In some applications, the operation parameters of sublingual implant 34—e.g., duty cycle, frequency, pulse duration or amplitude, of the sublingual implant's current, can be altered in response to how effective or painless the current generated by the implant is, and/or whether the implant disrupts the subject's sleep. This may be done with a wand (not shown), or with an input unit such as a keypad (not shown) on external device 24.

Feedback parameters for determining the efficacy and/or efficiency of the implant are obtained via receiving information from subject 22 himself, by way of sensors in external device 24, or in some applications, by feedback sensors (such as vibration or acceleration sensors, not shown) in sublingual implant 34.

It is noted that the number of sublingual implants in the schematic is by way of illustration and not limitation. One or more of the implants are typically injected into the subject in a minimally-invasive manner in an outpatient procedure. Typically, this is done through the lumen of a needle (not shown) using aseptic technique and with local anesthesia, as is known in the art. Other surgical procedures known in the art may also be used to place the implant in a muscle.

These one or more implants 34 can be configured to work in conjunction with other implants or independent of each other and/or external device 24.

In some applications, sublingual implant 34 is also configured to wirelessly receive power from external device 24, e.g., via a coil 48 coupled to circuitry 46. Other applications may use any one of, or a combination of, forms of wireless energy transfer, such as inductive coupling, RF, ultrasound, or other forms of wireless energy transfer.

In some applications, sublingual implant 34 is continuously provided with the necessary power to stimulate muscle via external device 34, or some other external device (not shown), in close enough proximity to wirelessly transfer power to the implant.

In some applications, sublingual implant 34 can temporarily store electrical power, e.g., by use of a battery or high-capacity capacitor (not shown), coupled by a wire (not shown) to the implant, or disposed within the implant.

In some applications, sublingual implant 34 has an internal battery that is periodically charged by external device 24 or some other external device (not shown), in close enough proximity to wirelessly transfer power to the implant.

For some applications, external device 24 operates substantially throughout the night, e.g., causing implant to drive current into sublingual muscle during most respiration cycles. Alternatively, external device 24 determines if the subject is sleeping, and only drives implant 34 during these periods (e.g., only during inspiration during sleep). Further alternatively, external device 24 determines if the subject is snoring, or exhibiting another respiration problem, and only drives the implant during such periods.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for treating obstructive sleep apnea, comprising:
an external device comprising:
a breathing sensor configured for placement outside of a body of a subject and to sense activity associated with breathing of the subject; and
a control unit, coupled to the breathing sensor, configured to generate a signal in response to the sensed activity associated with breathing; and
a sublingual implant, comprising:
a wireless receiver for receiving the signal;
two or more electrodes; and
circuitry coupled to the electrodes, the circuitry configured to drive the electrodes to apply current to a sublingual muscle site of the subject in response to the signal, in a plurality of successive periods of inspiration of the subject, even in the absence of a detection of apnea by the control unit.

2. The apparatus according to claim 1, wherein the breathing sensor is configured to sense a parameter selected from the group consisting of: airway pressure, snoring sounds, snoring motions, mechanical motion, and electrical activity associated with respiration.

3. The apparatus according to claim 1, wherein the sublingual implant is operative to vary at least one parameter of the applied current, the parameter selected from the group consisting of: a frequency of pulses of the current, an intensity of pulses of the current, and a duration of pulses of the current.

4. The apparatus according to claim 1, wherein the sublingual implant comprises an implant sensor configured to provide feedback to the external device.

5. The apparatus according to claim 1, wherein the control unit is configured to generate the signal during respective inspiratory phases of at least 30% of respiratory periods of the subject in a ten-minute period.

6. A method for treating obstructive sleep apnea, comprising:
identifying a subject as suffering from obstructive sleep apnea; and
in response, implanting, in a sublingual muscle of the subject, a sublingual implant that is configured to:
directly stimulate the muscle in a plurality of successive periods of inspiration of the subject, even in the absence of a detection of apnea; and
wirelessly receive energy from an external device to power the sublingual implant.

7. The method according to claim 6, further comprising configuring the external device to sense activity associated with breathing of the subject, and to generate a signal that causes the implant to stimulate the muscle during the inspiration periods.

8. The method according to claim 7, wherein configuring the external device further comprises placing the external device under a mattress.

9. The method according to claim 6, further comprising configuring the external device to sense breathing of the subject and to provide a signal to the sublingual implant to cause the implant to directly stimulate the muscle in response to the sensing of the breathing by the external device, during respective inspiratory phases of at least 30% of respiratory periods in an hour.

10. The method according to claim 6, wherein implanting the sublingual implant comprises sublingually injecting the implant.

11. A method for treating obstructive sleep apnea, comprising:
sublingually implanting a sublingual implant into a subject; and
configuring an external device to:
sense breathing of the subject;
provide a signal to the implant to cause the implant to directly stimulate a muscle in response to the sensing of the breathing by the external device, even in the absence of a detection of apnea, during respective inspiratory phases of at least 30% of respiratory periods in a ten-minute period; and
wirelessly transmit energy from the external device to the sublingual implant to power the sublingual implant.

12. A method for treating obstructive sleep apnea, comprising:
sublingually implanting a sublingual implant into a subject;
configuring the sublingual implant to provide feedback regarding breathing of the subject to an external device; and
configuring the external device to provide a signal to the implant to cause the implant to stimulate a muscle in response to the feedback provided by the sublingual implant.

13. The method according to claim 12, wherein configuring the external device comprises configuring the external device to sense breathing, and to provide the signal in response to the feedback provided by the sublingual implant and in response to the breathing sensed by the external device.

14. The method according to claim 13, wherein configuring the sublingual implant to provide feedback regarding the breathing of the subject comprises configuring the sublingual implant to provide feedback regarding snoring of the subject.
